(19) United States
(12) Patent Application Publication
(10) Pub. No.: US 2006/0247777 A1
(43) Pub. Date: Nov. 2, 2006

(54) Multiple bearing implant

(75) Inventor: Carl M. Stamp, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/118,188

(22) Filed: Apr. 29, 2005

(51) Int. Cl. A61F 2/44

(52) U.S. Cl. 623/17.14; 623/17.12

(57) ABSTRACT

A vertebral implant for interposition between two vertebral endplates comprises a first endplate component having a first exterior surface for engaging a first vertebral endplate and having a first interior surface. The implant further comprises a second endplate component having a second exterior surface for engaging a second vertebral endplate and having a second interior surface. The implant also comprises a plurality of movable bearing members attached to the second interior surface and movably engaged with the first interior surface.

### BACKGROUND

During the past thirty years, technical advances in the design of large joint reconstructive devices has revolutionized the treatment of degenerative joint disease, moving the standard of care from arthrodesis to arthroplasty. Progress in the treatment of vertebral disc disease, however, has come at a slower pace. Currently, the standard treatment for disc disease remains discectomy followed by vertebral fusion. While this approach may alleviate a patient's present symptoms, accelerated degeneration of adjacent discs is a frequent consequence of the increased motion and forces induced by fusion. Thus, reconstructing the degenerated intervertebral disc with a functional disc prosthesis to provide motion and to reduce deterioration of the adjacent discs may be a more desirable treatment option for many patients.

### SUMMARY

In one embodiment of the present disclosure, a vertebral implant for interposition between two vertebral endplates comprises a first endplate component having a first exterior surface for engaging a first vertebral endplate and having a first interior surface. The implant further comprises a second endplate component having a second exterior surface for engaging a second vertebral endplate and having a second interior surface. The implant also comprises a plurality of movable bearing members attached to the second interior surface and movably engaged with the first interior surface.

In another embodiment of the present invention, a vertebral implant for interposition between two vertebral endplates comprises a first endplate component having a first exterior surface for engaging a first vertebral endplate and having a first interior surface. The implant further comprises a second endplate component having a second exterior surface for engaging a second vertebral endplate and having a second interior surface. The implant also comprises a central body disposed between the first and second interior surfaces and a plurality of movable bearing members disposed between the central body and the first and second interior surfaces.

In another embodiment of the present disclosure, a system for preserving motion in a vertebral joint comprises a first means for attaching to a first vertebral endplate, a second means for attaching to a second vertebral endplate, and a plurality of third means disposed between the first and second means for providing rolling contact between the first means and the second means.

In another embodiment of the present disclosure, a vertebral implant for interposition between two vertebral endplates comprises a first endplate component having a first exterior surface for engaging a first vertebral endplate and having a first interior surface. The implant also comprises a second endplate component having a second exterior surface for engaging a second vertebral endplate and having a second interior surface. The second interior surface has a convex or concave portion. The implant also comprises a plurality of movable bearing members interposed between the second interior surface and the first interior surface.

In another embodiment of the present disclosure, a method for repairing an intervertebral joint comprises accessing an intervertebral disc space, removing at least a portion of a natural intervertebral disc from the intervertebral disc space, and inserting an intervertebral implant into the intervertebral disc space. The intervertebral implant comprises a first endplate component having a first exterior surface for engaging a first vertebral endplate and having a first interior surface and a second endplate component having a second exterior surface for engaging a second vertebral endplate and having a second interior surface. The second interior surface has a convex or concave portion. The implant further includes a plurality of movable bearing members interposed between the second interior surface and the first interior surface.

### BRIEF DESCRIPTION OF THE DRAWINGS

FIG. 1 is a side view of vertebral column with an intervertebral implant.

FIG. 2 is an exploded side sectional view of an intervertebral implant according to one embodiment of this disclosure.

FIG. 3 is an exploded side sectional view of an intervertebral implant according to another embodiment of this disclosure.

FIG. 4 is an exploded side sectional view of an intervertebral implant according to another embodiment of this disclosure.

FIG. 5 is an exploded side sectional view of an intervertebral implant according to another embodiment of this disclosure.

FIG. 6 is a side sectional view of an intervertebral implant according to another embodiment of this disclosure.

FIG. 7 is a side sectional view of an intervertebral implant according to another embodiment of this disclosure.

FIG. 8 is a top sectional view of an intervertebral implant according to another embodiment of this disclosure.

FIG. 9 is a top sectional view of an intervertebral implant according to another embodiment of this disclosure.

FIG. 10 is a top sectional view of an intervertebral implant according to another embodiment of this disclosure.

### DETAILED DESCRIPTION

The present invention relates generally to vertebral reconstructive devices, and more particularly, to a functional intervertebral disc prosthesis or implant. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

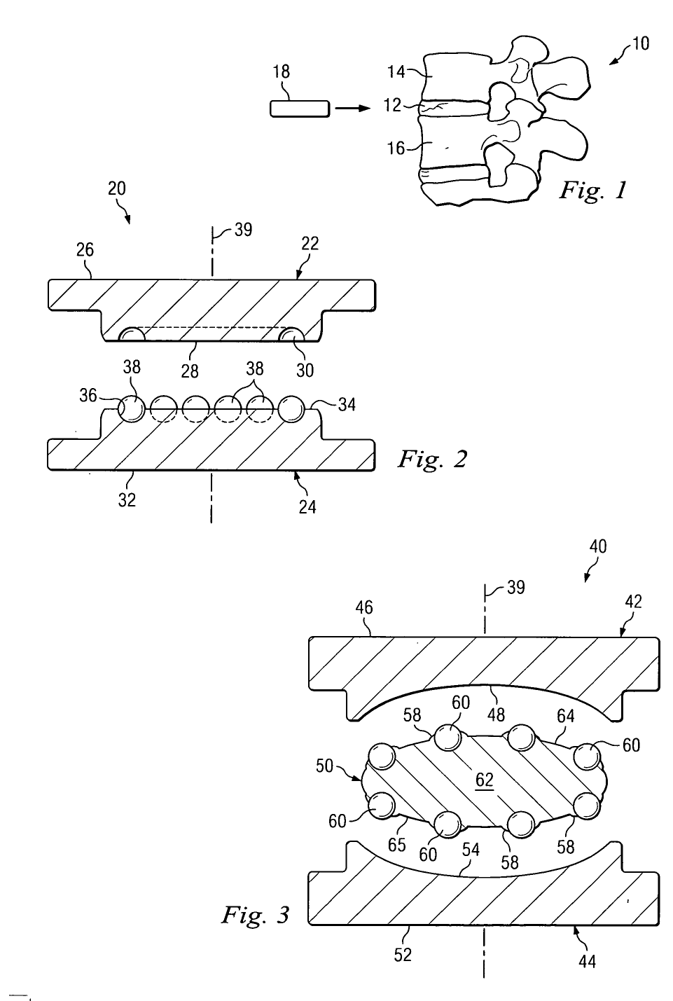

Referring first to FIG. 1, the numeral 10 refers to a vertebral joint which includes an intervertebral disc 12 extending between vertebrae 14, 16. The disc 12 may be partially or entirely removed and an intervertebral implant 18 may be inserted between the vertebrae 14, 16 to preserve motion within the joint 10. Although the illustration of FIG. 1 generally depicts the vertebral joint 10 as a lumbar vertebral joint, it is understood that the devices, systems, and methods of this disclosure may also be applied to all regions of the vertebral column, including the cervical and thoracic regions. Additionally, although the illustration of FIG. 1 generally depicts an anterior approach for insertion of the implant 18, other approaches including posterior, posterolateral, lateral, and anterolateral are also contemplated. Furthermore, the devices, systems, and methods of this disclosure may be used in non-spinal orthopedic applications.

Referring now to FIG. 2, an intervertebral implant 20 may be used as the prosthesis 18 of FIG. 1 according to one embodiment of the present disclosure. The intervertebral disc prosthesis 20 includes endplate components 22, 24. The endplate component 22 includes an exterior surface 26 and an interior surface 28. A race 30 may be formed in the surface 28. The race 30 may have a circular or oval path as viewed from a top sectional view. In this embodiment, the race 30 may be relatively smooth and may have a mirror surface finish. The endplate component 24 may have an exterior surface 32 and an interior surface 34. The surface 34 may further include a plurality of retaining components 36. The surfaces 28, 34, the race 30, and/or the retaining components 36 may be treated with any of various techniques to improve wear resistance such as ion-implantation, diamond or diamond-like coating, or other methods that make the surface harder than the original surface. The implant 20 may further include a plurality of bearing members 38 interposed between the interior surfaces 28, 34 to provide an articulating interface between the endplate components 22, 24. The bearing members 38 in this embodiment may be spherical ball bearings. Acceptable alternatives to spherical ball bearings may include a variety of roller bearings.

The endplate components 22, 24 and the bearing members 38 may be formed of any suitable biocompatible material including metals such as cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Certain polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

The exterior surfaces 26, 32 may include features or coatings (not shown) which enhance the fixation of the implanted prosthesis. For example, the surfaces may be roughened such as by chemical etching, bead-blasting, sanding, grinding, serrating, and/or diamond-cutting. All or portions of the exterior surfaces 26, 32 may receive a coating of a metallic substance which may be applied by sintering or by a spray coating such as a plasma spray. All or a portion of the exterior surfaces 26, 32 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes for initial fixation; ridges or keels to prevent migration in the lateral and anterior direction, for example; serrations or diamond cut surfaces; fins; posts; and/or other surface textures.

The prosthesis 20 may be assembled by inserting the bearing members 38 into the retaining components 36. Within the retaining components, the bearing members may be permitted to freely rotate. A portion of the bearing members 38 may extend from the retaining component, and the endplate component 22 may be positioned such that the race 30 engages the bearing members 38 in rolling contact. The assembled prosthesis 20 may be implanted into the vertebral joint 10 in the void created by the removed disc 12 such that the exterior surface 26 engages an endplate of the vertebral body 14 and the exterior surface 32 engages an endplate of the vertebral body 16. The prosthesis 20 may be implanted using any of the approaches described above.

In operation, the bearing members 38 may permit rotational motion of the endplate component 22 relative to the endplate component 24, thus promoting axial rotation at the vertebral joint 10 about a longitudinal axis 39. The bearing members 38 are permitted to roll within the race 30 as the endplates 22, 24 rotate relative to each other. This rolling motion of the bearing members 38 results in single point, rolling contact between the bearing members and the race 30. This minimum contact may reduce friction and the associated generation of wear debris, particularly as compared to surface contact and sliding motion.

In an alternative embodiment, the retaining component may be a retaining race, rather than individual retaining pockets. In this embodiment, the bearing members would still be retained by the retaining race, but would be permitted to roll along the circuit of the retaining race. In another alternative embodiment, a viscous fluid may be applied to the bearing members to provide lubrication. It is understood that the prosthesis may be implanted completely assembled, partially assembled or unassembled. All or portions of the assembly may be completed by the surgeon within the disc space.

Referring now to FIG. 3, an intervertebral implant 40 may be used as the prosthesis 18 of FIG. 1 according to another embodiment of the present disclosure. The intervertebral disc prosthesis 40 includes endplate components 42, 44. The endplate component 42 includes an exterior surface 46 and an interior surface 48. In this embodiment, the surface 48 may be relatively smooth and may have a mirror surface finish. The endplate component 44 may have an exterior surface 52 and an interior surface 54. The surface 54 may be relatively smooth and may have a mirror surface finish. The surfaces 48, 54 may be treated with any of various techniques to improve wear resistance such as ion-implantation, diamond or diamond-like coating, or other methods that make the surface harder than the original surface. A central body 50 extends between the interior surfaces 48, 54. The central body 50 may comprise a plurality of retaining components 58. A plurality of bearing members 60 may be located in the retaining components 58. The bearing members 60 in this embodiment may be spherical ball bearings. Acceptable alternatives to spherical ball bearings may include a variety of roller bearings.

The central body 50 may have an inner portion 62 and outer surfaces 64, 65. The central body 50 may have a generally circular cross-section as viewed from a plane perpendicular to the longitudinal axis 39. The inner portion 62 may be flexible and formed from one or more resilient materials which may have a lower modulus than the outer surfaces. Suitable materials may include polymeric elastomers such as polyolefin rubbers; polyurethanes (including polyetherurethane, polycarbonate urethane, and polyurethane with or without surface modified endgroups); copolymers of silicone and polyurethane with or without surface modified endgroups; silicones; and hydrogels. Polyisobutylene rubber, polyisoprene rubber, neoprene rubber, nitrile rubber, and/or vulcanized rubber of 5-methyl-1,4-hexadiene may also be suitable. In an alternative embodiment, the inner portion 62 may be rigid and formed of any of the materials described below for the outer surfaces or the endplate components.

The outer surfaces 64, 65 may also be formed of the resilient and flexible materials described above, but in the alternative, they may be modified, treated, coated or lined to enhance the wear resistant and articulating properties of the core component 50. These wear resistant and articulation properties may be provided by cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Polymer materials may also be used including any member of the PAEK family such as PEEK, carbon-reinforced PAEK, or PEKK; polysulfone; polyetherimide; polyimide; UHMWPE; and/or cross-linked UHMWPE. Polyolefin rubbers, polyurethanes, copolymers of silicone and polyurethane, and hydrogels may also provide wear resistance and articulation properties. Wear resistant characteristics may also or alternatively be provided to the outer surfaces 64, 65 by modifications such as cross-linking and metal ion implantation.

The endplate components 42, 44 and the bearing members 60 may be formed of any suitable biocompatible material including metals such as cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

The exterior surfaces 46, 52 may include features or coatings (not shown) which enhance the fixation of the implanted prosthesis. For example, the surfaces may be roughened such as by chemical etching, bead-blasting, sanding, grinding, serrating, and/or diamond-cutting. All or portions of the exterior surfaces 46, 52 may receive a coating of a metallic substance which may be applied by sintering or by a spray coating such as a plasma spray. All or a portion of the exterior surfaces 46, 52 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes for initial fixation; ridges or keels to prevent migration in the lateral and anterior direction, for example; serrations or diamond cut surfaces; fins; posts; and/or other surface textures.

The prosthesis 40 may be assembled by inserting the bearing members 60 into the retaining components 58. Within the retaining components 58, the bearing members 60 may be permitted to freely rotate. A portion of the bearing members 60 may extend from the retaining component. The central body 50 may then be inserted between the interior surfaces 48, 54 such that the bearing members 60 engage the surfaces 48, 54 in rolling contact. The assembled prosthesis 40 may be implanted into the vertebral joint 10 in the void created by the removed disc 12 such that the exterior surface 46 engages an endplate of the vertebral body 14 and the exterior surface 52 engages an endplate of the vertebral body 16. The prosthesis 40 may be implanted using any of the approaches described above.

In operation, the central body 50 may allow a variable center of rotation to permit flexion-extension and lateral bending motions. The bearing members 60 may provide a rolling interface between the central body 50 and the interior surfaces 48, 54 which permits rotational motion, translational motion, and bending motion of the endplate component 42 relative to the endplate component 44. This, in turn, may permit axial rotation, lateral bending, and flexion-extension motion at the vertebral joint 10. The rolling motion of the bearing members 60 results in single point contact between the bearing members and interior surfaces 48, 54. This minimum contact may reduce friction and the associated generation of wear debris, particularly as compared to sliding, shear-generating motion. The flexible nature of the core component 50 may also reduce wear caused by cross-shearing or by articulation in flexion-extension and lateral bending motions.

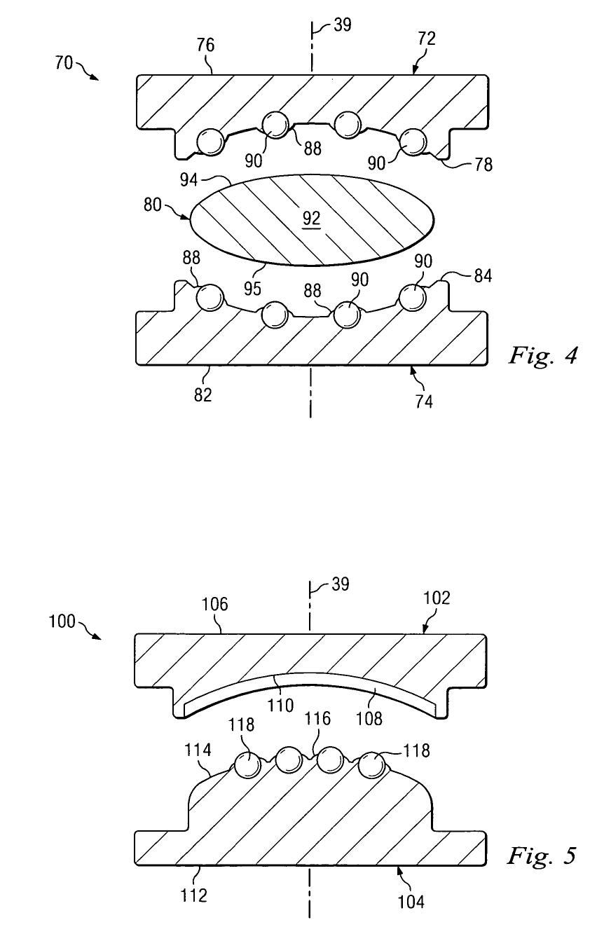

Referring now to FIG. 4, an intervertebral implant 70 may be used as the prosthesis 18 of FIG. 1 according to another embodiment of the present disclosure. The intervertebral disc prosthesis 70 includes endplate components 72, 74. The endplate component 72 includes an exterior surface 76 and an interior surface 78. The endplate component 44 may have an exterior surface 82 and an interior surface 84. A central body 80 extends between the interior surfaces 78, 84. The interior surfaces 78, 84 may comprise a plurality of retaining components 88. A plurality of bearing members 90 may be located in the retaining components 88. The bearing members 90 in this embodiment may be spherical ball bearings. Acceptable alternatives to spherical ball bearings may include a variety of roller bearings.

The central body 80 may have an inner portion 92 and outer surfaces 94, 95. The central body 80 may have a generally circular cross-section as viewed from a plane perpendicular to a longitudinal axis 39. The inner portion 92 and the outer surfaces 94, 95 may be formed of any of the materials described above for inner portion 62 and outer surfaces 64, 65, respectively. The surfaces 94, 95 may be treated with any of various techniques to improve wear resistance such as ion-implantation, diamond or diamond-like coating, or other methods that make the surface harder than the original surface. The endplate components 72, 74 and the bearing members 90 may be formed of any suitable material including those materials described above for endplate components 42, 44. The exterior surfaces 76, 82 may include features or coatings (not shown) which enhance the fixation of the implanted prosthesis such as those described above for surfaces 46, 52.

The prosthesis 70 may be assembled by inserting the bearing members 90 into the retaining components 88. Within the retaining components 88, the bearing members 90 may be permitted to freely rotate. A portion of the bearing members 90 may extend from the retaining component. The central body 80 may then be inserted between the interior surfaces 78, 84 such that the bearing members 80 engage the surfaces 94, 95 in rolling contact. The assembled prosthesis 70 may be implanted into the vertebral joint 10 in the void created by the removed disc 12 such that the exterior surface 76 engages an endplate of the vertebral body 14 and the exterior surface 82 engages an endplate of the vertebral body 16. The prosthesis 70 may be implanted using any of the approaches described above.

In operation, the central body 80 may allow a variable center of rotation to permit flexion-extension and lateral bending motions. The bearing members 90 may provide a rolling interface between the central body 80 and the interior surfaces 78, 84 which permits rotational motion, translational motion, and bending motion of the endplate component 72 relative to the endplate component 74. This, in turn, may permit axial rotation, lateral bending, and flexion-extension motion at the vertebral joint 10. The rolling motion of the bearing members 90 results in single point contact between each of the bearing members and central body 80. This minimum contact may reduce friction and the associated generation of wear debris, particularly as compared to sliding motion. A flexible core component 80 may also reduce wear caused by cross-shearing or by articulation in flexion-extension and lateral bending motions.

Referring now to FIG. 5, an intervertebral implant 100 may be used as the prosthesis 18 of FIG. 1 according to one embodiment of the present disclosure. The intervertebral disc prosthesis 100 includes endplate components 102, 104. The endplate component 102 includes an exterior surface 106 and a concave interior surface 108. A recessed portion 110 may be formed in the surface 108. In this embodiment, the recessed portion 110 may be relatively smooth and may have a mirror surface finish. The recessed portion 110 may have a generally circular or oval shape. The endplate component 104 may have an exterior surface 112 and a convex interior surface 114. The surface 114 may be relatively smooth and may have a mirror surface finish. The surface 114 may further include a plurality of retaining components 116. The surfaces 108, 104, the recessed portion 110, and/or the retaining components 116 may be treated with any of various techniques to improve wear resistance such as ion-implantation, diamond or diamond-like coating, or other methods that make the surface harder than the original surface. The implant 100 may further include a plurality of bearing members 118 interposed between the interior surfaces 108, 114 to provide an articulating interface between the components 102, 104. The bearing members 108 in this embodiment may be spherical ball bearings. Acceptable alternatives to spherical ball bearings may include a variety of roller bearings.

The endplate components 102, 104 and the bearing members 118 may be formed of any suitable biocompatible material including those described above for endplate components 22, 24. The exterior surfaces 106, 112 may include features or coatings (not shown) which enhance the fixation of the implanted prosthesis such as those described above for surfaces 26, 32.

The prosthesis 20 may be assembled by inserting the bearing members 118 into the retaining components 116. Within the retaining component, the bearing members may be permitted to freely rotate. A portion of the bearing members 118 may extend from the retaining components 116, and the endplate component 102 may be positioned such that the recessed portion 110 engages the bearing members 118 in rolling contact. The assembled prosthesis 100 may be implanted into the vertebral joint 10 in the void created by the removed disc 12 such that the exterior surface 106 engages an endplate of the vertebral body 14 and the exterior surface 112 engages an endplate of the vertebral body 16. The prosthesis 100 may be implanted using any of the approaches described above.

In operation, the bearing members 118 may move within the boundaries of the recessed portion 110. The bearing members 118 may provide a rolling interface between the interior surfaces 108, 114 which permits rotational motion, translational motion, and bending motion of the endplate component 102 relative to the endplate component 104. This, in turn, may permit axial rotation, lateral bending, and flexion-extension motion at the vertebral joint 10. The rolling motion of the bearing members 118 results in single point contact between the bearing members and interior surfaces 108, 114. This minimum contact may reduce friction and the associated generation of wear debris, particularly as compared to sliding motion.

Referring now to FIG. 6, an intervertebral implant 120 may be used as the prosthesis 18 of FIG. 1 according to another embodiment of the present disclosure. The intervertebral disc prosthesis 120 includes endplate components 122, 124; a central body 126; and a plurality of bearing members 128. This prosthesis 120 may be substantially similar to the structure, assembly, and operation of the prosthesis 70 described above, except for the differences to be described. The prosthesis 120 further includes a circumferential sheath 130 that extends between the endplate components 122, 124 and encapsulates the central body 126 between the endplate components. The sheath 130 may be filled with a lubricating fluid 132 which may lubricate the bearing members 128 and promote motion of the central body 126. The sheath 130 may serve to retain both the fluid 132 and any generated wear debris. Further the sheath 130 may serve as a barrier preventing unwanted tissue or fluid from contacting the central body 126 and the bearing members 128.

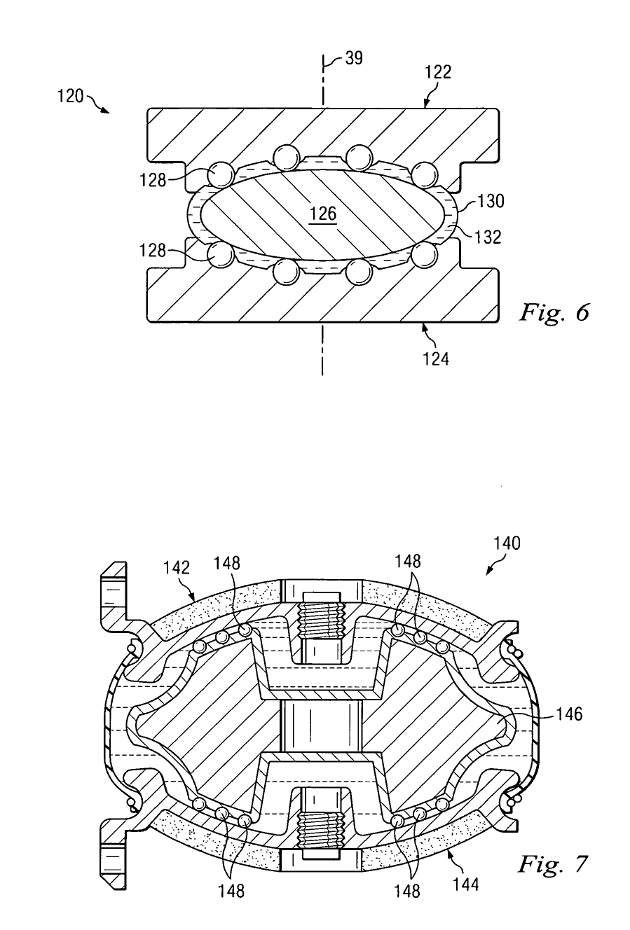

Referring now to FIG. 7, an intervertebral implant 140 may be used as the prosthesis 18 of FIG. 1 according to another embodiment of the present disclosure. Except for the differences to be described, the implant 140 may be substantially similar to an implant disclosed in U.S. patent application Ser. No. 09/924,298, entitled “Implantable Joint Prosthesis” which is incorporated herein by reference. Specifically, the implant 140 includes opposing shells or endplate components 142, 144 and a central body 146. In this embodiment, a plurality of bearing members 148 may be rotatably attached to the central body 146 to reduce the shear forces and the friction at the interface between the central body 146 and the endplate components 142, 144 during translation, lateral bending, flexion-extension, or rotational motion. In an alternative embodiment, the bearing members 148 may be rotatably attached to the endplate components 142, 144 rather than the central body 146.

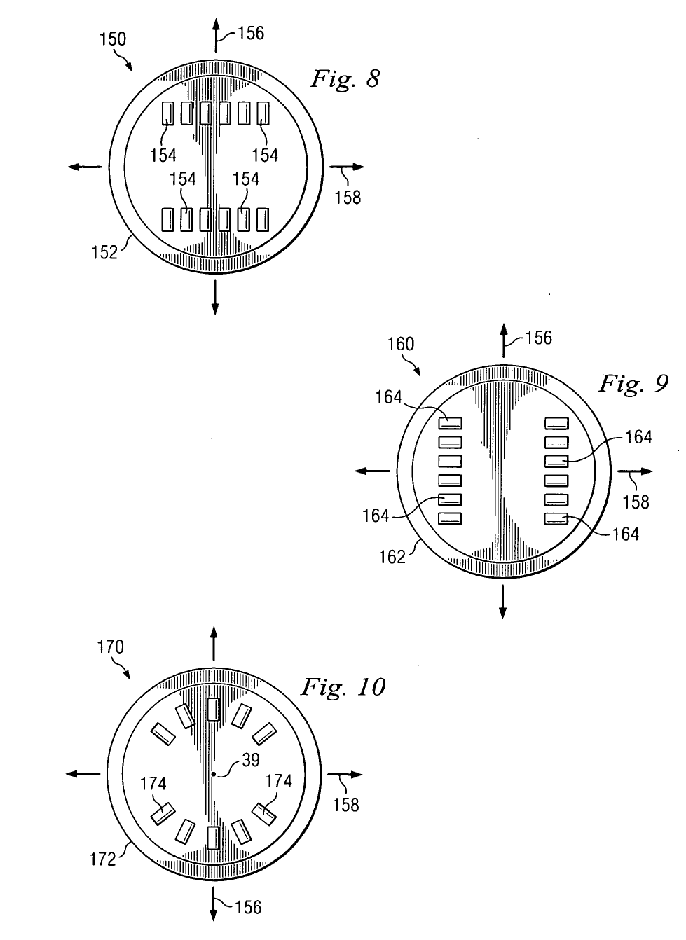

Referring now to FIG. 8, an intervertebral implant 150 may be used as the prosthesis 18 of FIG. 1 according to another embodiment of the present disclosure. The implant 150 includes a lower endplate component 152 fitted with a plurality of bearing members 154 which in this embodiment may be roller bearings. An upper endplate component (not shown) may also be provided, with the bearing members 154 serving as an interface between the upper and lower bearings. The endplate component 152 may be substantially similar to any of the endplate components 24, 74, or 104, except for the differences to be described. The bearing members 154 may be arranged to promote certain motions and limit other types of motion. For example, to restrict translation of the lower endplate component 152 with respect to the upper component in an anterior-posterior direction 156, the roller bearings 154 may be arranged such that rolling occurs in a lateral direction 158, thus permitting lateral translation and/or lateral bending. The roller bearings 154, in this configuration, may limit anterior-posterior translation or flexion-extension motion.

Referring now to FIG. 9, an intervertebral implant 160 may be used as the prosthesis 18 of FIG. 1 according to another embodiment of the present disclosure. The implant 160 includes a lower endplate component 162 fitted with a plurality of bearing members 164 which in this embodiment may be roller bearings. An upper endplate component (not shown) may also be provided, with the bearing members 164 serving as an interface between the upper and lower bearings. The endplate component 162 may be substantially similar to any of the endplate components 24, 74, or 104, except for the differences to be described. The bearing members 164 may be arranged to promote certain motions and limit other types of motion. For example, to restrict translation of the lower endplate component 162 with respect to the upper component in the lateral direction 158, the roller bearings 164 may be arranged such that rolling occurs in the anterior-posterior direction 156, thus permitting anterior-posterior translation and/or flexion-extension motion. The roller bearings 164, in this configuration, may limiting lateral translation or lateral bending.

Referring now to FIG. 10, an intervertebral implant 170 may be used as the prosthesis 18 of FIG. 1 according to another embodiment of the present disclosure. The implant 170 includes a lower endplate component 172 fitted with a plurality of bearing members 174 which in this embodiment may be roller bearings. An upper endplate component (not shown) may also be provided, with the bearing members 174 serving as an interface between the upper and lower bearings. The endplate component 172 may be substantially similar to any of the endplate components 24, 74, or 104, except for the differences to be described. The bearing members 174 may be arranged to promote certain motions and limit other types of motion. For example, the roller bearings 174 may be arranged such that rolling occurs about the longitudinal axis 39, thus limiting anterior-posterior translation and flexion-extension motion. The roller bearings 164, in this configuration, may limit, although not necessarily entirely restrict, lateral translation, lateral bending, anterior-posterior translation, or flexion-extension motion.

In alternative embodiments, other arrangements of roller bearings, spherical ball bearings, races, or rolling surfaces may be arranged to promote or limit certain types of motion. In still other alternative embodiments, the motion limiting bearings arrangement may be rotatably attached to an upper endplate component or a central body.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “rostral,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A vertebral implant for interposition between two vertebral endplates comprising:
a first endplate component having a first exterior surface for engaging a first vertebral endplate and having a first interior surface;
a second endplate component having a second exterior surface for engaging a second vertebral endplate and having a second interior surface; and
a plurality of movable bearing members movably coupled to the second interior surface and movably engaged with the first interior surface.

2. The vertebral implant of claim 1 wherein the first interior surface comprises a race and the plurality of movable bearing members are movably engaged with the race.

3. The vertebral implant of claim 1 wherein the second interior surface has a convex shape.

4. The vertebral implant of claim 1 wherein the second interior surface has a concave shape.

5. The vertebral implant of claim 1 wherein the first interior surface has a convex shape.

6. The vertebral implant of claim 1 wherein the first interior surface has a concave shape.

7. The vertebral implant of claim 1 wherein the first interior surface further comprises a recessed portion and the plurality of movable bearing members are movably engaged with the recessed portion.

8. The vertebral implant of claim 1 wherein the plurality of movable bearing members comprises a plurality of spherical bearings.

9. The vertebral implant of claim 1 wherein the plurality of movable bearing members comprises a plurality of roller bearings.

10. The vertebral implant of claim 1 wherein plurality of movable bearing members permit flexion-extension motion and limit lateral bending.

11. The vertebral implant of claim 1 wherein the plurality of movable bearing members permit lateral bending and limit flexion-extension motion.

12. The vertebral implant of claim 1 wherein the plurality of movable bearing members permit rotational motion of the first endplate component with respect to the second endplate component.

13. The vertebral implant of claim 1 wherein the plurality of movable bearing members includes at least three rotatable bearing members.

14. A vertebral implant for interposition between two vertebral endplates comprising:
a first endplate component having a first exterior surface for engaging a first vertebral endplate and having a first interior surface;
a second endplate component having a second exterior surface for engaging a second vertebral endplate and having a second interior surface;
a central body disposed between the first and second interior surfaces; and
a plurality of movable bearing members disposed between the central body and the first and second interior surfaces.

15. The vertebral implant of claim 14 wherein a portion of the plurality of movable bearing members is in movable contact with the first interior surface and another portion of the plurality of movable bearing members is in movable contact with the second interior surface.

16. The vertebral implant of claim 14 wherein the plurality of movable bearing members are movably coupled to the central body.

17. The vertebral implant of claim 14 wherein the first and second interior surfaces are concave.

18. The vertebral implant of claim 14 wherein the first and second exterior surfaces are convex and the first and second interior surfaces are concave.

19. The vertebral implant of claim 14 further comprising a flexible sheath extending between the first and second endplate components, wherein the flexible sheath and the first and second interior surfaces define an enclosure housing the central body.

20. The vertebral implant of claim 19 wherein the enclosure contains a lubricating fluid.

21. A system for preserving motion in a vertebral joint, the system comprising:
a first means for attaching to a first vertebral endplate;
a second means for attaching to a second vertebral endplate; and
a plurality of third means disposed between the first and second means for providing rolling contact between the first means and the second means.

22. A vertebral implant for interposition between two vertebral endplates comprising:
a first endplate component having a first exterior surface for engaging a first vertebral endplate and having a first interior surface;
a second endplate component having a second exterior surface for engaging a second vertebral endplate and having a second interior surface, wherein the second interior surface includes a convex or concave portion; and
a plurality of movable bearing members interposed between the second interior surface and the first interior surface.

23. A method for repairing an intervertebral joint, the method comprising:
accessing an intervertebral disc space;
removing at least a portion of a natural intervertebral disc from the intervertebral disc space; and
inserting an intervertebral implant into the intervertebral disc space, wherein the intervertebral implant comprises:
a first endplate component having a first exterior surface for engaging a first vertebral endplate and having a first interior surface;
a second endplate component having a second exterior surface for engaging a second vertebral endplate and having a second interior surface, wherein the second interior surface includes a convex or concave portion; and
a plurality of movable bearing members interposed between the second interior surface and the first interior surface.
